SEQUENCE LISTING (1 sequence)

SEQ ID NO: 1 - DNA, 4896 bp, Clostridium sporogenes <s300> Dickert, S., Pierik, A.J. and Buckel, W. Molecular characterization of (r)-phenyllactate dehydratase and its activating component A from Clostridium sporogenes Laboratorium fuer Mikrobiologie 44 1 49-60 2002-04-01 AF420489 2016-07-26
aattccctttaacagatacaggtaaaattaagaggcatgaactaaaaaaatgctttgaaaagaagtttgaattaagacaatctatttaaattaataataaatatattaaattaacaataaatatattaaattaacaataaatctatttaaggaggctttttttatggaaaacaatacaaatatgtttagtggagtaaaggttattgaattagcaaattttatagctgctccagcagcaggtagattttttgctgatggtggtgcagaggtaataaaaattgaatcacctgctggagatcctttaagatatactgctccttcagaaggaagaccattaagccaagaagaaaatactacttatgatttggaaaatgcaaataaaaaagcaatagtattaaatcttaaaagcgaaaaaggtaaaaagatattacatgaaatgttagcagaagcagatatattattaactaattggagaacaaaggctttagttaaacaaggattagactatgaaacactaaaagaaaaatatcctaaattagtttttgcacaaataactggttatggtgaaaaaggaccagataaagatcttccaggctttgattatactgcatttttcgctagaggcggtgtttcaggtactctttatgaaaaaggaactgtgcctccaaatgttgttccaggacttggagaccatcaagctgggatgtttttagcagcgggtatggcaggagctttatataaagcaaaaacaacaggacaaggagataaagtaacagtaagtttaatgcatagtgctatgtatggactaggtattatgatacaagctgctcaatataaagatcatggattagtatatccgataaatcgtaatgaaactccaaatccttttatagtttcatataaatctaaggatgattactttgttcaagtatgtatgccaccatatgatgttttctatgatagatttatgaccgctttaggaagagaagatttagttggagacgaaagatacaataaaatagaaaatttaaaagatggacgtgctaaggaagtatacagtataatcgaacaacaaatggttacaaagacaaaggatgaatgggataacatatttagagatgcagacattccatttgctatcgcacaaacttgggaagatttattagaagatgaacaagcttgggcaaatgattatttgtataagatgaaatatccaacaggaaacgaaagagcattagtaagacttccagtattctttaaagaagcaggattaccagaatataatcaatcaccacaaatagcagaaaatactgtagaagttttaaaagaaatgggatatacagaacaagagattgaggaattagaaaaagataaagatataatggtaaggaaggaaaaataatggcagacatttatactatgggtgtagacataggttcaactgcatcaaaaacagtagtattaaaaaatggtaaagaaattgtaagtcaagcagtaataagtgtaggggccggaacaagtggccccaagagagctatagattctgtattaaaagatgctaaattatccattgaagatttagactatattgtatccactggatatggaagaaatagtttcgattttgctaacaaacaaatttctgaattaagttgtcatgcaaaaggggtctatttcgataacaataaagctagaacagttattgatataggcggacaagatattaaagtattaaaattagcggatagtggaagacttttaaactttataatgaatgataaatgtgctgcaggaacgggacgatttttagatgtaatgtctagagtaatagaagttccagttgatgagttaggaaaaaaagcattagaaagcaaaaatccttgtactattagttctacctgtacagtatttgcagagtcagaagtaatttctcaacttgcaagaggagttaaaactgaagatttgatagcaggaatttgtaaatctgtagcatcaagagtggctagccttgcaaagagaagtggtatagaagaattagtagttatgagtggaggagtagctaaaaatataggtgtagtaaaggcaatggaagcagaattgggaagagacatatatatatctaaaaattctcaattaaatggagcattgggagcaagtctatacgcttatgaaagttttcaaaaagaaaggagctaaaaacatgagtgatagaaataaggaagtaaaagaaaaaaaggcaaagcattatcttagagagattactgcaaagcattacaaagaagctctcgaagcaaaagaaaggggagaaaaggttggttggtgtgcatctaacttcccacaagaaatagctacaacattgggggtaaaagttgtttatccagaaaatcatgcagcagctgtagcagctagagggaatggacaaaatatgtgtgaacatgctgaggctatgggtttttctaatgatgtatgtggttatgcaagagtaaatttagctgttatggacataggtcatagtgaagatcaaccaatacctatgccagactttgtactttgctgtaataacatttgtaatcaaatgattaaatggtatgagcatatagcaaaaactttagatataccaatgattcttatagatataccatacaatacagaaaatactgtttcacaagatagaattaaatatattagagcacaatttgatgatgcaataaaacaattggaagaaataacaggcaaaaaatgggatgaaaataaatttgaagaagttatgaaaatatcccaagaaagtgcaaaacaatggttaagagcagcatcctatgcaaagtataaaccttcaccatttagcggatttgatttatttaatcatatggctgtagcagtttgtgcaagaggtacacaagaagctgcagatgcatttaagatgttagcagatgaatatgaggagaatgtaaaaactggaaaatccacttataggggagaagaaaaacaacgtatattatttgaagggattgcctgttggccatatttgagacataaattaactaagcttagtgaatatggtatgaacgtaactgcaactgtatacgcagaagcctttggtgttatatatgagaatatggatgaattaatggctgcttataataaagttcctaattcaattagttttgaaaacgcattaaaaatgagattaaatgctgttacaagcactaatacagaaggtgctgttattcatataaatagaagctgtaaattatggagtggatttttatatgagctagcaagaagattagaaaaggaaacaggaattcctgtagtatcatttgatggggaccaggcagacccaagaaatttctcagaagctcaatatgatactagaattcaaggacttaatgaagtaatggttgctaaaaaggaggctgaataagatgtcaaattcagataaattttttaatgactttaaggatattgtagaaaatcctaaaaaatatataatgaagcatatggaacaaactggacaaaaggctataggatgtatgccattatatactcctgaggaacttgtattagctgctggaatgtttccagtaggggtatggggaagcaatacagaactttcaaaagctaaaacatatttcccagcatttatttgttcaatattacaaacaacattggaaaatgcattaaatggagaatatgatatgttatctggtatgatgattacaaattattgtgattcattaaaatgcatgggacaaaattttaaactaaccgttgaaaatattgagtttatcccagtaacagttccacaaaatagaaaaatggaagctggaaaagagtttttaaaaagtcaatataaaatgaatattgagcaattagaaaagatttctggtaataaaataacagatgaatctttagaaaaagctatagaaatatatgatgaacacagaaaagtaatgaatgacttttcaatgttagcatcaaaatatccaggtataataacaccaactaaacgtaattatgttatgaaatctgcttattatatggataaaaaagaacatactgaaaaagttagacaattaatggatgaaattaaagctatagaaccaaaaccatttgaaggaaagagagttataactacaggtataattgcagattcagaagatttacttaaaatattagaagaaaataatatagctatagttggtgatgatatagcacatgaatctagacaatatagaacattgactccagaagcgaacacaccaatggataggttagctgagcaatttgctaatagagaatgtagtactttatatgatcctgaaaagaaaaggggtcaatatatagtagaaatggctaaagagagaaaagcagatggaattatatttttcatgacaaaattctgtgacccagaggaatatgattatccacaaatgaaaaaggattttgaagaagcaggcattccacatgtactaatagaaactgatatgcaaatgaaaaattatgaacaagctagaactgcaattcaggctttttcagaaacactttaataaaagttttcaatatttactgtaaactttattaattgaaacattgatttctcttctctttctataaaataatatttatatttaaaaaagttatgtttagatggatgaaaggaaatcaatgttcatataaattaacaaattcattaatatattaggagggatataatgtttttcacagaacaacatgaacttattagaaaattagcaagagattttgcagagcaggaaatagagcctattgcagatgaagtagataaaactgccgagttccctaaagaaattgtgaaaaaaatggcccaaaatggtttttttggaataaaaatgcctaaagaatatggtggagctg

(19) United States
(12) Patent Application Publication
(10) Pub. No.: US 2020/0316023 A1
(43) Pub. Date: Oct. 8, 2020

(54) Method Of Producing Bacterially Derived Indole-3-Propionic Acid And Compositions Comprising Same

(71) Applicant: The Procter & Gamble Company, Cincinnati, OH (US)

(72) Inventors: Arvind Venkataraman, Mason, OH (US); Chelsay Lynn Brewster, Hamilton, OH (US); Kimberly Conner Kozak, Lebanon, OH (US); Lijuan Li, Lebanon, OH (US); Jason Allen Stamper, Covington, KY (US)

(21) Appl. No.: 16/838,045

(22) Filed: Apr. 2, 2020

Related U.S. Application Data
(60) Provisional application No. 62/827,998, filed on Apr. 2, 2019.

(51) Int. Cl. A61K 31/405; C12P 13/00; A61K 35/747; A61K 35/745; A61K 35/744; A61K 35/74

(52) U.S. Cl. CPC A61K 31/405; C12P 13/001; A61K 35/74; A61K 35/745; A61K 35/744; A61K 35/747

(57) ABSTRACT

Described herein is a method for producing indole-3-propionic acid and other indole derivatives via bacterial fermentation and compositions of the same. The method comprises adding bacteria having a nucleic acid sequence with at least 80% homology to the nucleic acid sequence of SEQ ID NO: 1 to a liquid fermentation medium; fermenting at about 36° C. under anaerobic conditions; adding a dehydrating agent; and dehydrating to obtain a fermentate powder comprising indole-3-propionic acid and other indole derivatives.

### FIELD

Described herein is a method of producing bacterially derived indole-3-propionic acid and other indole derivatives. Also described herein are compositions comprising indole-3-propionic acid and other indole derivatives that can help support brain health and/or nervous system function.

### INCORPORATION BY REFERENCE OF THE SEQUENCE LISTING

This application contains, as a separate part of disclosure, a Sequence Listing in computer-readable form (filename: 15497P_ST25.txt; Size: 7,196 bytes; Created: Apr. 2, 2019) which is incorporated by reference herein in its entirety.

### BACKGROUND

Practicing good nutrition can be challenging. Some people seek supplements to provide additional nutrients to improve their health and wellness, including maintaining healthy brain function. The brain is particularly susceptible to oxidative stress due to its high rate of oxygen consumption, its large content of polyunsaturated fatty acids and regional high iron levels, and its proportionately low antioxidant capacity. It is known that oxidative stress can cause reduced neurogenesis and increased neuronal death. It has been shown that cognitive impairment is related to oxidative stress and an efficient antioxidant system can preserve the cognitive function in older adults.

Indole-3-propionic acid (“IPA”) is a neuroprotective antioxidant that may improve mood, cognition, and/or maintain healthy brain function and nervous system in humans. IPA is made by the gut microbiome in the colon and crosses the intestinal epithelium and blood brain barrier to enter the brain. In the brain, IPA has been shown to play a protective role as an antioxidant, thereby protecting the structure & function of neurons. It is believed that the antioxidant property of IPA can play a key role in promoting brain health. It is well known that the consumption of IPA by mouth can increase IPA levels in situ. (See Kaufmann SHE. 2018. Indole propionic acid: a small molecule links between gut microbiota and tuberculosis. Antimicrob Agents Chemother 62:e00389-18; Niebler G. NCT01898884: Safety and Pharmacology Study of VP 20629 in Adults With Friedreich's Ataxia (2018).

Despite the growing appreciation of the beneficial effects of IPA on brain health, IPA is only commercially produced in a chemically synthesized form. However, an increasing number of consumers have an interest in product ingredients, including their origin, and prefer supplements from natural sources. The direct ingestion of chemically synthesized IPA may not be preferred by these natural-seeking consumers. Furthermore, along with IPA, other indole derivatives such as indole-3-acetic acid, indole-3-acrylic acid, and indole-3-lactic acid are also emerging as providing positive health benefits. However, chemically synthesized forms of IPA only deliver pure IPA.

Thus, there is a need for a naturally derived means of providing a combination of indole derivatives, within which IPA would be a major component, in order to promote brain health and/or nervous system function.

### SUMMARY

Described herein is composition comprising: (a) a fermentate comprising bacterially derived indole-3-propionic acid and other indole derivatives; and (b) an excipient, carrier, and/or diluent.

Described herein is a method of producing indole-3-propionic acid and other indole derivatives comprising: (a) adding bacteria having a nucleic acid sequence with at least 80% homology to the nucleic acid sequence of SEQ ID NO: 1 to a liquid fermentation medium to form a bacteria solution; (b) fermenting the bacteria solution at about 36° C. under anaerobic conditions; (c) adding a dehydrating agent; and (d) dehydrating to obtain a fermentate powder comprising indole-3-propionic acid and other indole derivatives.

Described herein is a composition comprising: a fermentate comprising bacterially derived indole-3-propionic acid and other indole derivatives and an excipient, carrier, and/or diluent; wherein the fermentate is obtained by a process comprising (a) adding bacteria having a nucleic acid sequence with at least 80% homology to the nucleic acid sequence of SEQ ID NO: 1 to a liquid fermentation medium to form a bacteria solution; (b) fermenting the bacteria solution at about 36° C. under anaerobic conditions; (c) adding a dehydrating agent; and (d) dehydrating to obtain the fermentate.

Described herein is a method of promoting brain health by delivering antioxidant nutrients to the brain by administering a composition comprising: (a) a fermentate comprising bacterially derived indole-3-propionic acid and other indole derivatives; and (b) an excipient, carrier, and/or diluent.

### DETAILED DESCRIPTION

Consumers are looking for effective and natural ways of supplementing their diets with IPA in order to promote brain and mental well-being. Described herein is a method of producing IPA and other indole derivatives through bacterial fermentation and compositions of the same. It has been found that bacteria fermentation in the presence of tryptophan and other components such as amino acids, vitamins, and trace metals can produce naturally derived IPA and other indole derivatives that can be dried into a fermentate powder without negatively impacting stability of the IPA or other indole derivatives. As used herein, “other indole derivatives” refers to tryptophan derived indole metabolites including indole-3-acrylic acid, indole-3-lactic acid, and indole-3-acetic acid.

As used herein, the terms “administer,” “administering,” and “administration,” refer to any method which, in sound medical practice, delivers the composition to a subject in such a manner as to provide a therapeutic effect.

As used herein, “anaerobic conditions” refer to any growth or nutrient conditions that exclude the presence of oxygen (e.g., less than about 1 ppm free oxygen, preferably less than about 0.1 ppm free oxygen, more preferably from about 0 to about 1 ppm free oxygen).

As used herein, the abbreviation “CFU” (“colony forming units”) designates the number of bacterial cells revealed by microbiological counts on agar plates, as will be commonly understood in the art.

As used herein, “fermentation” refers to a process by which microorganisms metabolize raw materials.

As used herein, “fermentate” refers to the isolated solids after removal of water from a fermentation medium with or without prior removal of the bacteria.

The terms “microbes” and “microorganisms” are used interchangeably herein to refer to bacteria. The terms “microbiome”, “microbiota”, and “microbial habitat” are used interchangeably herein and can refer to the ecological community of microorganisms that live on or in a subject's body. Microbiomes can exist on or in many, if not most, parts of the subject. Some non-limiting examples of habitats of microbiome can include: body surfaces, body cavities, body fluids, the gut, the colon, skin surfaces and pores, vaginal cavity, umbilical regions, conjunctival regions, intestinal regions, the stomach, the nasal cavities and passages, the gastrointestinal tract, the urogenital tracts, saliva, mucus, and feces.

As used herein, the term “prebiotic” refers to chemicals and/or ingredients that can affect the growth and/or activity of microorganisms in a subject or host (e.g., can allow for specific changes in the composition and/or activity in the microbiome) and can confer a health benefit on the subject.

The term “probiotic” as used herein can mean one or more live microorganisms (e.g., bacteria or yeast) which, when administered appropriately, can confer a health benefit on the subject. “Nucleic acid sequence” and “nucleotide sequence” as used herein refer to an oligonucleotide or polynucleotide, and fragments or portions thereof, and to DNA or RNA of genomic or synthetic origin which may be single- or double-stranded and represent the sense or antisense strand. The nucleic acid sequence can be made up of adenine, guanine, cytosine, thymine, and uracil (A, T, C, G, and U) as well as modified versions (e.g. N6-methyladenosine, 5-methylcytosine, etc.).

The terms “subject” refers to any animal subject, including humans, laboratory animals, livestock, and household pets.

As used herein, the articles “a” and “an” are understood to mean one or more of the material that is claimed or described, for example, “an active ingredient” or “a probiotic”.

The composition can contain, consist of, or consist essentially of, the essential elements and limitations of the invention described herein, as well as any additional or optional ingredients, components, or limitations described herein or otherwise useful in compositions intended for use or consumption by a subject.

A method of producing IPA and other indole derivatives can comprise the steps of:
a. adding bacteria capable of producing IPA and other indole derivatives to a liquid fermentation medium to form a bacteria solution;
b. fermenting the bacteria solution under anaerobic conditions to form a fermented bacteria solution;
c. terminating fermentation;
d. optionally concentrating the bacteria solution by reducing the water content (e.g. via reverse osmosis, tray drying, microfiltration, nanofiltration, and combinations thereof);
e. adding a dehydrating agent; and
f. dehydrating to obtain a fermentate powder comprising indole-3-propionic acid and other indole derivatives.

Bacteria capable of producing IPA and other indole derivatives include, for example, Clostridium sporogenes, Peptostreptococcus anaerobius, Clostridium cadaveris, Clostridium boltae, and any other bacteria having a nucleic acid sequence that is substantially homologous to the nucleic acid sequence of SEQ ID NO: 1 (Table 1), which encodes the phenyllactate dehydratase gene cluster (fldL, fldI, and fldABC).

TABLE 1
DNA sequence

| SEQ ID | |
|---|---|
| NO | Nucleotide Sequence |
| 1 | AATTCCCTTTAACAGATACAGGTAAAATTAAGAGGCATGAACTAAAAAAA |
| | TGCTTTGAAAAGAAGTTTGAATTAAGACAATCTATTTAAATTAATAATAAA |
| | TATATTAAATTAACAATAAATATATTAAATTAACAATAAATCTATTTAAGG |
| | AGGCTTTTTTTATGGAAAACAATACAAATATGTTTAGTGGAGTAAAGGTTA |
| | TTGAATTAGCAAATTTTATAGCTGCTCCAGCAGCAGGTAGATTTTTTGCTGA |
| | TGGTGGTGCAGAGGTAATAAAAATTGAATCACCTGCTGGAGATCCTTTAAG |
| | ATATACTGCTCCTTCAGAAGGAAGACCATTAAGCCAAGAAGAAAATACTA |
| | CTTATGATTTGGAAAATGCAAATAAAAAAGCAATAGTATTAAATCTTAAAA |
| | GCGAAAAAGGTAAAAAGATATTACATGAAATGTTAGCAGAAGCAGATATA |
| | TTATTAACTAATTGGAGAACAAAGGCTTTAGTTAAACAAGGATTAGACTAT |
| | GAAACACTAAAAGAAAAATATCCTAAATTAGTTTTTGCACAAATAACTGGT |
| | TATGGTGAAAAAGGACCAGATAAAGATCTTCCAGGCTTTGATTATACTGCA |
| | TTTTTCGCTAGAGGCGGTGTTTCAGGTACTCTTTATGAAAAAGGAACTGTG |
| | CCTCCAAATGTTGTTCCAGGACTTGGAGACCATCAAGCTGGGATGTTTTTA |
| | GCAGCGGGTATGGCAGGAGCTTTATATAAAGCAAAAACAACAGGACAAGG |
| | AGATAAAGTAACAGTAAGTTTAATGCATAGTGCTATGTATGGACTAGGTAT |
| | TATGATACAAGCTGCTCAATATAAAGATCATGGATTAGTATATCCGATAAA |
| | TCGTAATGAAACTCCAAATCCTTTTATAGTTTCATATAAATCTAAGGATGAT |
| | TACTTTGTTCAAGTATGTATGCCACCATATGATGTTTTCTATGATAGATTTA |
| | TGACCGCTTTAGGAAGAGAAGATTTAGTTGGAGACGAAAGATACAATAAA |
| | ATAGAAAATTTAAAAGATGGACGTGCTAAGGAAGTATACAGTATAATCGA |
| | ACAACAAATGGTTACAAAGACAAAGGATGAATGGGATAACATATTTAGAG |
| | ATGCAGACATTCCATTTGCTATCGCACAAACTTGGGAAGATTTATTAGAAG |
| | ATGAACAAGCTTGGGCAAATGATTATTTGTATAAGATGAAATATCCAACAG |
| | GAAACGAAAGAGCATTAGTAAGACTTCCAGTATTCTTTAAAGAAGCAGGA |
| | TTACCAGAATATAATCAATCACCACAAATAGCAGAAAATACTGTAGAAGTT |
| | TTAAAAGAAATGGGATATACAGAACAAGAGATTGAGGAATTAGAAAAAGA |
| | TAAAGATATAATGGTAAGGAAGGAAAAATAATGGCAGACATTTATACTAT |
| | GGGTGTAGACATAGGTTCAACTGCATCAAAAACAGTAGTATTAAAAAATG |
| | GTAAAGAAATTGTAAGTCAAGCAGTAATAAGTGTAGGGGCCGGAACAAGT |
| | GGCCCCAAGAGAGCTATAGATTCTGTATTAAAAGATGCTAAATTATCCATT |
| | GAAGATTTAGACTATATTGTATCCACTGGATATGGAAGAAATAGTTTCGAT |
| | TTTGCTAACAAACAAATTTCTGAATTAAGTTGTCATGCAAAAGGGGTCTAT |
| | TTCGATAACAATAAAGCTAGAACAGTTATTGATATAGGCGGACAAGATATT |
| | AAAGTATTAAAATTAGCGGATAGTGGAAGACTTTTAAACTTTATAATGAAT |
| | GATAAATGTGCTGCAGGAACGGGACGATTTTTAGATGTAATGTCTAGAGTA |
| | ATAGAAGTTCCAGTTGATGAGTTAGGAAAAAAAGCATTAGAAAGCAAAAA |
| | TCCTTGTACTATTAGTTCTACCTGTACAGTATTTGCAGAGTCAGAAGTAATT |
| | TCTCAACTTGCAAGAGGAGTTAAAACTGAAGATTTGATAGCAGGAATTTGT |
| | AAATCTGTAGCATCAAGAGTGGCTAGCCTTGCAAAGAGAAGTGGTATAGA |
| | AGAATTAGTAGTTATGAGTGGAGGAGTAGCTAAAAATATAGGTGTAGTAA |
| | AGGCAATGGAAGCAGAATTGGGAAGAGACATATATATATCTAAAAATTCT |
| | CAATTAAATGGAGCATTGGGAGCAAGTCTATACGCTTATGAAAGTTTTCAA |
| | AAAGAAAGGAGCTAAAAACATGAGTGATAGAAATAAGGAAGTAAAAGAA |
| | AAAAAGGCAAAGCATTATCTTAGAGAGATTACTGCAAAGCATTACAAAGA |
| | AGCTCTCGAAGCAAAAGAAAGGGGAGAAAAGGTTGGTTGGTGTGCATCTA |
| | ACTTCCCACAAGAAATAGCTACAACATTGGGGGTAAAAGTTGTTTATCCAG |
| | AAAATCATGCAGCAGCTGTAGCAGCTAGAGGGAATGGACAAAATATGTGT |
| | GAACATGCTGAGGCTATGGGTTTTTCTAATGATGTATGTGGTTATGCAAGA |
| | GTAAATTTAGCTGTTATGGACATAGGTCATAGTGAAGATCAACCAATACCT |
| | ATGCCAGACTTTGTACTTTGCTGTAATAACATTTGTAATCAAATGATTAAAT |
| | GGTATGAGCATATAGCAAAAACTTTAGATATACCAATGATTCTTATAGATA |
| | TACCATACAATACAGAAAATACTGTTTCACAAGATAGAATTAAATATATTA |
| | GAGCACAATTTGATGATGCAATAAAACAATTGGAAGAAATAACAGGCAAA |
| | AAATGGGATGAAAATAAATTTGAAGAAGTTATGAAAATATCCCAAGAAAG |
| | TGCAAAACAATGGTTAAGAGCAGCATCCTATGCAAAGTATAAACCTTCACC |
| | ATTTAGCGGATTTGATTTATTTAATCATATGGCTGTAGCAGTTTGTGCAAGA |
| | GGTACACAAGAAGCTGCAGATGCATTTAAGATGTTAGCAGATGAATATGA |
| | GGAGAATGTAAAAACTGGAAAATCCACTTATAGGGGAGAAGAAAAACAAC |
| | GTATATTATTTGAAGGGATTGCCTGTTGGCCATATTTGAGACATAAATTAA |
| | CTAAGCTTAGTGAATATGGTATGAACGTAACTGCAACTGTATACGCAGAAG |
| | CCTTTGGTGTTATATATGAGAATATGGATGAATTAATGGCTGCTTATAATA |
| | AAGTTCCTAATTCAATTAGTTTTGAAAACGCATTAAAAATGAGATTAAATG |
| | CTGTTACAAGCACTAATACAGAAGGTGCTGTTATTCATATAAATAGAAGCT |
| | GTAAATTATGGAGTGGATTTTTATATGAGCTAGCAAGAAGATTAGAAAAGG |
| | AAACAGGAATTCCTGTAGTATCATTTGATGGGGACCAGGCAGACCCAAGA |
| | AATTTCTCAGAAGCTCAATATGATACTAGAATTCAAGGACTTAATGAAGTA |
| | ATGGTTGCTAAAAAGGAGGCTGAATAAGATGTCAAATTCAGATAAATTTTT |
| | TAATGACTTTAAGGATATTGTAGAAAATCCTAAAAAATATATAATGAAGCA |
| | TATGGAACAAACTGGACAAAAGGCTATAGGATGTATGCCATTATATACTCC |
| | TGAGGAACTTGTATTAGCTGCTGGAATGTTTCCAGTAGGGGTATGGGGAAG |
| | CAATACAGAACTTTCAAAAGCTAAAACATATTTCCCAGCATTTATTTGTTCA |
| | ATATTACAAACAACATTGGAAAATGCATTAAATGGAGAATATGATATGTTA |
| | TCTGGTATGATGATTACAAATTATTGTGATTCATTAAAATGCATGGGACAA |
| | AATTTTAAACTAACCGTTGAAAATATTGAGTTTATCCCAGTAACAGTTCCA |
| | CAAAATAGAAAAATGGAAGCTGGAAAAGAGTTTTTAAAAAGTCAATATAA |
| | AATGAATATTGAGCAATTAGAAAAGATTTCTGGTAATAAAATAACAGATG |
| | AATCTTTAGAAAAAGCTATAGAAATATATGATGAACACAGAAAAGTAATG |
| | AATGACTTTTCAATGTTAGCATCAAAATATCCAGGTATAATAACACCAACT |
| | AAACGTAATTATGTTATGAAATCTGCTTATTATATGGATAAAAAAGAACAT |
| | ACTGAAAAAGTTAGACAATTAATGGATGAAATTAAAGCTATAGAACCAAA |
| | ACCATTTGAAGGAAAGAGAGTTATAACTACAGGTATAATTGCAGATTCAGA |
| | AGATTTACTTAAAATATTAGAAGAAAATAATATAGCTATAGTTGGTGATGA |
| | TATAGCACATGAATCTAGACAATATAGAACATTGACTCCAGAAGCGAACA |
| | CACCAATGGATAGGTTAGCTGAGCAATTTGCTAATAGAGAATGTAGTACTT |
| | TATATGATCCTGAAAAGAAAAGGGGTCAATATATAGTAGAAATGGCTAAA |
| | GAGAGAAAAGCAGATGGAATTATATTTTTCATGACAAAATTCTGTGACCCA |
| | GAGGAATATGATTATCCACAAATGAAAAAGGATTTTGAAGAAGCAGGCAT |
| | TCCACATGTACTAATAGAAACTGATATGCAAATGAAAAATTATGAACAAGC |
| | TAGAACTGCAATTCAGGCTTTTTCAGAAACACTTTAATAAAAGTTTTCAAT |
| | ATTTACTGTAAACTTTATTAATTGAAACATTGATTTCTCTTCTCTTTCTATAA |
| | AATAATATTTATATTTAAAAAAGTTATGTTTAGATGGATGAAAGGAAATCA |
| | ATGTTCATATAAATTAACAAATTCATTAATATATTAGGAGGGATATAATGT |
| | TTTTCACAGAACAACATGAACTTATTAGAAAATTAGCAAGAGATTTTGCAG |
| | AGCAGGAAATAGAGCCTATTGCAGATGAAGTAGATAAAACTGCCGAGTTC |
| | CCTAAAGAAATTGTGAAAAAAATGGCCCAAAATGGTTTTTTTGGAATAAAA |
| | ATGCCTAAAGAATATGGTGGAGCTG |

Bacteria comprise nucleic acid sequences having a particular degree of homology or identity to other bacteria. The terms “identity,” “homology,” and “homologous” as used herein refer to a degree of complementarity or shared similarity with other nucleotide sequences. There may be partial homology or complete homology (i.e., identical sequences). A nucleotide sequence which is partially complementary, i.e., “substantially homologous” or “substantially identical” to a nucleic acid sequence is one that at least partially inhibits a completely complementary sequence from hybridizing to a target nucleic acid sequence.

In some aspects, bacteria can comprise a nucleic acid sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% homologous or identical to the nucleic acid sequence of SEQ ID NO: 1.

In some aspects, bacteria comprising the nucleic acid sequence of SEQ ID NO: 1 can be a probiotic or a probiotic bacterium.

The bacteria require a fermentation medium in which to ferment and produce the IPA and derivatives thereof. The fermentation medium can be any suitable medium that can allow microorganism growth and fermentation. In some aspects, the fermentation medium can be a standard amino acid complete medium. In some aspects, the fermentation medium can comprise water, an amino acid composition, a vitamin, a salt, and a mineral. In some aspects, the fermentation medium can comprise water, an amino acid composition, a vitamin, a salt, a carbohydrate, and a mineral.

In some aspects, the amino acid composition can comprise one or more amino acids. Non-limiting examples of amino acids can include glutamine, lysine, cysteine, methionine, aspartic acid, leucine, valine, alanine, arginine, glycine, tyrosine, tryptophan, phenylalanine, histidine, leucine, isoleucine, and combinations thereof. The amino acids should include those that are suitable for IPA production. Examples of amino acid compounds that can be used include cysteine HCl, L-glycine, L-valine, L-leucine, L-isoleucine, L-methionine, L-histidine, L-arginine, L-phenylalanine, L-tyrosine, and L-tryptophan. The amount of amino acids will vary depending on the amount of IPA desired to be produced. In some aspects, the fermentation medium can comprise from about 8 to about 10,000 μg/mL of amino acids, alternatively from about 10 to about 8,000 μg/mL, alternatively from about 25 to about 5,000 μg/mL, alternatively from about 50 to about 1,000 μg/mL, alternatively from about 100 to about 500 μg/mL.

In some aspects, the ratio of other amino acids to tryptophan should be greater than 1:1. It is believed that the other amino acids should be present at a concentration greater than that of tryptophan in order to improve the yield of IPA and other indole derivatives.

In some aspects, the fermentation medium can comprise one or more salts. Salt can be added to the fermentation medium to improve the viability of the bacteria and/or can increase the yield of IPA and other indole derivatives. Non-limiting examples of salts can include calcium carbonate, ammonium sulfate, magnesium sulfate, monopotassium phosphate, dipotassium phosphate, magnesium chloride, sodium bicarbonate, and combinations thereof. The amount of salt added to the fermentation medium should be sufficient to obtain the desired result of improving the viability and/or increasing the yield IPA and derivatives thereof. In some aspects, the fermentation medium can comprise from about 10 to about 5,000 mg/L salt, alternatively from about 20 to about 1,000 mg/L, alternatively from about 50 to about 800 mg/L, alternatively from about 75 to about 500 mg/L.

In some aspects, the fermentation medium can comprise a carbohydrate. Carbohydrates can include polysaccharides, oligosaccharides, disaccharides, monosaccharides, and combinations thereof. Non-limiting examples of suitable carbohydrates can include maltose, gum acacia, and glucose. In some aspects, the fermentation medium can comprise from about 2 to about 40 mM carbohydrate, alternatively from about 5 to about 30 mM, alternatively from about 10 to about 25 mM.

In some aspects, the fermentation medium can comprise one or more vitamins. Non-limiting examples of vitamins can include vitamin B1, vitamin B2, vitamin B3, vitamin B5, vitamin B6, vitamin B7, vitamin B9, vitamin B12, and combinations thereof. In some aspects, the fermentation medium can comprise a vitamin solution, such as Wolfe's Vitamin Solution or Vitamin Supplement ATCC® MD-VSTM (commercially available from ATCC, Manassas, Va.).

In some aspects, the fermentation medium can also include one or more trace elements, such as zinc, manganese, and nickel. In some aspects, the fermentation medium can comprise a trace element solution, such as Wolfe's Mineral Solution or Trace Mineral Supplement ATCC® MD-TMS™ (commercially available from ATCC, Manassas, Va.).

The fermentation medium can have a pH of from about 5 to about 8, alternatively from about 5.5 to about 7.5, alternatively from about 6 to about 7. Without being limited by theory it is believed that the pH can be selected to increase the yield of IPA and derivatives thereof.

The fermentation medium can be prepared by any known methods in the art.

The bacteria can be added anaerobically to the fermentation medium to form a bacteria solution. The number of CFU of bacteria added to the fermentation medium can vary based on the type of bacteria used and the amount of IPA desired to be produced. In some aspects, fermentation can be performed at an initial cell concentration of between 1E3 to about 1E9 CFU/mL of fermentation medium, preferably from 1E5 to about 1E8 CFU/mL, more preferably about 1E8 CFU/mL.

The bacteria solution can be maintained under conditions that permit optimal bacteria growth. For example, the bacteria solution can be maintained at a temperature of from about 25° C. to about 45° C., preferably from about 30° C. to about 40° C., more preferably about 36° C., under anaerobic conditions.

In some aspects, the bacteria should be permitted to ferment for a sufficient period of time to produce the desired amount of IPA and derivatives thereof. In some aspects, the bacteria solution is incubated at about 36° C. under anaerobic conditions for about 24 to about 48 hours, alternatively for about 2 hours to about 72 hours, alternatively from about 4 hours to about 48 hours, alternatively from about 8 hours to about 36 hours, alternatively for about 12 hours to about 36 hours.

In some aspects, fermentation may be concluded by one or more process steps in which the bacteria are inactivated or physically removed. The bacteria can be inactivated by heating (typically between 30 minutes and 3 hours at a temperature of between about 65° C. to about 93° C.) or by treatment with a proteolytic enzyme, such as papain or bromelain. Alternatively, the fermented bacteria solution can be centrifuged to form a bacteria pellet and a supernatant solution and the bacteria pellet can be discarded, leaving the supernatant solution containing the IPA and other indole derivatives, which can be dehydrated to form a fermentate. Alternatively, the fermented bacteria solution can be passed through a membrane filter to remove the bacteria.

In some aspects, the solution can be homogenized after fermentation in order to form a more uniform product. Methods of homogenization are known in the art, and can be performed, for example, by a homogenization pump, shearing pump, or a blender. It is preferred that the solution be dehydrated after fermentation. Methods for dehydrating solutions are well known in the art and can include freeze-drying, spray drying, open air drying, spray granulation, and drum drying. The preferred dehydration method is spray drying or freeze-drying.

Optionally, prior to dehydration, the residual water content in the fermented bacteria solution can be substantially reduced by, for example, a combination of reverse osmosis, tray drying, microfiltration, and/or nanofiltration. Methods of reverse osmosis, tray drying, microfiltration, and nanofiltration can be performed using methods and equipment well known in the art.

Freeze-drying can be performed using methods well known in the art. In particular, the freeze-drying process can consist of a thermal treatment step followed by a drying step. In the thermal treatment step, the vials can be held at about 20° C. for about 30 minutes, followed by about 0° C. for about 80 minutes, and then at about −25° C. for about 60 minutes. The freezing can be performed at about −25° C. (condenser at about −50° C.) and vacuum at about 200 mTorr. In the drying step, the vials can be held at about −25° C. for a total of about 1800 minutes at about 200 mTorr and then the temperature can be ramped up to about 4° C. and vials held at about 4° C. for about 60 minutes at about 200 mTorr.

Spray-drying can be performed using methods and equipment well known in the art. Preferably, a spray dryer, such as a Büchi Mini Spray Dryer B-191 available from Büchi Labortechnik AG, Flawil, Switzerland, or equivalent is used. The spray dryer can be run with an inlet temperature of about 185° C. and the flow rate of the pump feeding the dryer can be set to achieve an exit temperature of about 100° C. The spry dryer unit can be operated with a two-fluid atomizer. The atomizer can deliver the liquid feed into the dryer. The airflow rate can be set to a volumetric flow rate of about 35 cubic meters per hour.

Prior to dehydration, a dehydrating agent can be added to the solution to facilitate drying and/or improve stability. In some aspects, a dehydrating agent can be a cryoprotectant such as inositol, sorbitol, mannitol, trehalose, glucose, sucrose, corn syrup, DMSO, starches and/or modified starches of all types, Polyvinylpyrrolidone (PVP), maltose, or other mono and disaccharides, and combinations thereof. The dehydrating agent can be utilized at any level suitable for facilitating drying, for instance from about 2 to about 10 wt %, alternatively from about 3 to about 8 wt %, alternatively from about 4 to about 6 wt %. Preferably, the dehydrating agent is a modified starch, such as Hi-Cap® 100 modified food starch derived from waxy maize (commercially available from Ingredion, Westchester, Ill.).

The solution can be dehydrated to a residual water content of less than about 15 wt %, alternatively less than about 10 wt %, alternatively less than about 5 wt %. Alternatively, and especially where the residual water content is greater than about 5 wt %, additional agents may be included that reduce water activity to a value of equal or less than about 0.75, alternatively equal or less than about 0.7, alternatively equal or less than about 0.65, alternatively equal or less than about 0.55, alternatively equal or less than about 0.40.

After dehydrating, a powder fermentate comprising IPA and derivatives thereof is formed which can then be incorporated into a dosage form or other form suitable for administration.

Also described herein is a composition comprising a fermentate comprising bacterially derived IPA and other indole derivatives and a physiologically, pharmaceutically, or nutritionally acceptable excipient, carrier and/or diluent. In some aspects, the composition can comprise one or more strains or species of bacteria in combination with the bacterially derived IPA and other indole derivates. In some aspects, the fermentate can further comprise tryptophan.

The composition can comprise a fermentate. The fermentate can be obtained by the process described above. In some aspects, the fermentate can comprise IPA, other indole derivatives, and tryptophan. In some aspects, the fermentate can optionally comprise inactive bacteria having a nucleic acid sequence with at least 80% homology to the nucleic acid sequence of SEQ ID NO: 1.

In some aspects, the composition can comprise from about 1 mg to about 2 g of the fermentate, alternatively from about 10 mg to about 1.5 g, alternatively from about 25 mg to about 1 g. In some aspects, the composition can comprise from about 1 mg to about 500 mg of the fermentate, alternatively from about 15 mg to about 250 mg, alternatively from about 50 mg to about 150 mg.

In one aspect, the composition can comprise from about 0.01% to about 90% of the fermentate, alternatively from about 0.1% to about 85%, alternatively from about 1% to about 80%, alternatively from about 2.5% to about 75%, alternatively from about 5% to about 60%, alternatively from about 10% to about 50%, alternatively from about 15% to about 25%, all by weight of the composition.

In some aspects, the composition can comprise bacteria from about 1×E3 to about 1×E13 CFU/g of fermentate.

In some aspects, the composition can comprise from about 0.1 mg to about 20 mg IPA, alternatively from about 1 mg to about 8 mg, alternatively from about 2 mg to about 6 mg. In some aspects the composition can comprise from about 0.01% to about 10% IPA, alternatively from about 1% to about 8%, alternatively from about 2% to about 6%, all by weight of the composition.

In some aspects, the composition can comprise from about 0.1 mg/g to about 20 mg/g IPA, alternatively from about 1 mg/g to about 8 mg/g, alternatively from about 2 mg/g to about 6 mg/g.

In some aspects, the composition can comprise from about 0.01 mg to about 10 mg other indole derivatives, alternatively from about 0.1 mg to about 8 mg, alternatively from about 1 mg to about 6 mg. In some aspects, the composition can comprise from about 0.01% to about 10% other indole derivatives, alternatively from about 1% to about 8%, alternatively from about 2% to about 6%, all by weight of the composition.

In some aspects, the composition can comprise from about 0.01 mg to about 10 mg tryptophan, alternatively from about 0.1 mg to about 8 mg, alternatively from about 1 mg to about 5 mg. In some aspects the composition can comprise from about 0.1% to about 10% tryptophan, alternatively from about 1% to about 8%, alternatively from about 2% to about 5%, all by weight of the composition. In some aspects, the fermentate can comprise from about 0.1 mg/g to about 0.5 mg/g of tryptophan, preferably 0.2 mg/g.

In some aspects, the fermentate can comprise glucose. The amount of glucose present in the fermentate can depend upon when the fermentation reaction is terminated, which in turn will be a function of the growth phase of the bacterium, and IPA yield.

In some aspects, the composition can comprise one or more bacteria. In some aspects, the one or more bacteria can be probiotic bacteria. Suitable probiotic bacteria can include Bifidobacterium bifidum, Bifidobacterium breve, Bifidobacterium infantis, Bifidobacterium longum, Streptococcus cremoris, Streptococcus diacetylactis, Streptococcus lactis, Streptococcus thermophilus, Lactobacillus acidophilus, Lactobacillus bifidus, Lactobacillus bulgaricus, Lactobacillus casei, Lactobacillus delbruekii, Lactobacillus crispatis, Lactobacillus fermentii, Lactobacillus gasseri, Lactobacillus helveticus, Lactobacillus johnsonii, Lactobacillus lactis, Lactobacillus plantarum, Lactobacillus rhamnosus, Lactobacillus paracasei, Lactobacillus reuteri, Lactobacillus salivarius, Lactobacillus thermophilus, Lactococcus lactis, and combinations thereof.

In some aspects, the one or more bacteria can comprise C. sporogenes. In some aspects, the C. sporogenes can be inactive.

In some aspects, the one or more bacteria are Bifidobacterium infantis, particularly B. infantis 35624.

In some aspects, the composition can comprise an excipient, carrier, and/or diluent. Nutritionally acceptable excipients, carriers or diluents include, but are not limited to, those suitable for human or animal consumption and those that are used standardly in the food industry. Typical nutritionally acceptable excipients, carriers or diluents are familiar to the skilled person in the art.

Examples of such suitable excipients for the various different compositions described herein, in some aspects, are found in the “Handbook of Pharmaceutical Excipients, 2nd Edition, (1994), Edited by A Wade and P J Weller. Acceptable carriers or diluents, in some aspects, are described, for example, in Remington's Pharmaceutical Sciences, Mack Publishing Co. (A. R. Gennaro edit. 1985). Such suitable carriers include, but are not limited to, lactose, methyl cellulose, magnesium stearate, and the like. Such suitable diluents include, but are not limited to water, ethanol, and glycerol.

The choice of pharmaceutical excipient, carrier, or diluent is selected with regard to the intended route of administration and standard pharmaceutical or nutraceutical practice. Such compositions, in some aspects, may comprise, in addition to the excipient, carrier or diluent, additional ingredients. Such additional ingredients include, but are not limited to, any suitable binder(s), lubricant(s), suspending agent(s), coating agent(s), solubilizing agent(s), preservatives, dyes, flavoring agent(s), and/or suspending agents.

Examples of suitable binders include, but are not limited to, starch, gelatin, and natural sugars. Such natural sugars include, but are not limited to, glucose, anhydrous lactose, free-flow lactose, beta-lactose, corn sweeteners, and natural and/or synthetic gums, such as acacia, tragacanth or sodium alginate, carboxymethyl cellulose and polyethylene glycol. Examples of suitable lubricants include, but are not limited to, sodium oleate, sodium stearate, magnesium stearate, sodium benzoate, sodium acetate, sodium chloride and the like. Preservatives, stabilizers, dyes, and flavoring agents, in some aspects, are also provided in the composition. Examples of preservatives include, but are not limited to, sodium benzoate, sorbic acid, and esters of p-hydroxybenzoic acid. In some aspects, suspending agents may also be present in the composition.

In some aspects, the composition can optionally comprise one or more active ingredients. Non-limiting examples of active ingredients can include vitamins, minerals, prebiotics, glycans (e.g., as decoys that would limit specific bacterial/viral binding to the intestinal wall), melatonin, and combinations thereof. Non-limiting examples of vitamins can include vitamin C, vitamin D, vitamin E, vitamin K1, Vitamin K3, vitamin B1, vitamin B3, folic acid, vitamin B12, vitamin B2, vitamin B3, vitamin B6, vitamin B7, and pantothenic acid (vitamin B5). Non-limiting examples of minerals can include calcium, selenium, magnesium, iron, iodide, zinc, copper, manganese, chromium, molybdenum, beta-carotene, and combinations thereof.

The term “prebiotic” as used herein can be a general term to refer to chemicals and/or ingredients that can affect the growth and/or activity of microorganisms in a subject or host (e.g., can allow for specific changes in the composition and/or activity in the microbiome) and can confer a health benefit on the subject. Prebiotics include, but are not limited to, complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, and xylooligosaccharides (XOS). Prebiotic substrates, such as these, improve the colonization and survival of the bacteria in vivo. Prebiotics, in some aspects, are selectively fermented, e.g., in the colon.

Prebiotics, in various aspects, are found in foods (e.g., acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. In some aspects, prebiotics are administered in other forms (e.g. capsule or dietary supplement).

The active ingredients can be at levels above, below, and/or equal to the recommended daily allowance (“RDA”), depending on the particular active ingredient. Exemplary RDA values for numerous nutritional compounds are listed in 21 CFR 101 and further RDA values are also published by the Institute of Medicine of the National Academy of Science.

In some aspects, the active ingredient is present in an amount from about 0.01 to about 50% by weight, with respect to the total weight of the composition. In some aspects, the active ingredient can be present in an amount from about 0.1 to about 40% by weight, alternatively from about 1 to about 30%, alternatively from about 3 to about 25%, alternatively from about 5 to about 20%. In some aspects, the active ingredient is present in an amount of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, or 50%.

The composition can optionally comprise one or more herbal ingredients. Non-limiting examples of herbal ingredients can include rosemary (leaf), ginger, lemon balm, green tea, holy basil, oregano, thyme, ashwagandha, bacopa, and combinations thereof. In some aspects, the composition comprises ashwagandha. In some aspects, the herbal ingredient can be whole herbs or plant parts, extracts, powders, concentrates, or combinations thereof. In some aspects, the herbal ingredient can be supercritical extracts and/or hydroalcoholic extracts. As used herein, the term “supercritical extraction” refers to the technique in which hydrophobic compounds can be extracted from samples utilizing a supercritical fluid. The solvation power of a supercritical fluid is increased as the pressure and temperature are increased above their critical points, producing an effective solvent for the isolation of hydrophobic molecules. In some aspects, the herbal ingredients can be fermented using methods known to one of skill in the art. A particularly suitable fermentation method is described in U.S. Pat. No. 6,806,069, which is herein incorporated by reference in its entirety. The fermented herbal ingredients can be prepared by collecting the supernatants of the herbal fermentations and drying the mixture by any known method in the art, such as spray-drying. The culture media can contain ingredients selected from the group consisting of organic milled soy, Saccharomyces cerevisiae (organic yeast: active and inactive), organic maltodextrin, organic gum acacia, organic orange peel, organic lemon peel, organic carrot powder, organic alfalfa powder, Lactobacilli (L. acidophilus, L. bifidus, L. rhamnosus) and enzymes (deactivated), and combinations thereof. The fermented herbal ingredients can contain all or some of the ingredients from the culture media.

In some aspects, the composition can comprise from about 0.1 to about 10% of the one or more herbal ingredients, alternatively from about 1 to about 8%, alternatively from about 2 to about 6%, all by weight of the composition.

In some aspects, the composition can be substantially free of vitamins, minerals, and/or herbs which inhibit IPA production. In some aspects, the composition can be substantially free of Vitamin B2, selenium, and/or Vitamin B6. As used herein, “substantially free of” means containing less than about 0.1%, by weight of the composition, alternatively less than about 0.05% alternatively less than about 0.01%, alternatively less than about 0.001%.

The composition can be in any dosage form known in the art. Some non-limiting examples of dosage forms can include topical, capsule, pill or tablet, gummy, soft chew, panned chew, sachet, gel, liquid, bulk powder for reconstitution or a drink prepared from bulk powder, and the like. In some aspects, the composition can be incorporated into a form of food and/or drink. Non-limiting examples of food and drinks where the composition is incorporated can include bars, shakes, juices, beverages, frozen food products, fermented food products, and cultured dairy products such as yogurt, yogurt drink, cheese, acidophilus drinks, and kefir.

In some aspects, the composition may be in the form of a dietary supplement or a pharmaceutical composition. As used herein, the term “dietary supplement” refers to a composition intended to supplement a diet of food and water, where the diet is sufficient to support life.

In some aspects, the composition can comprise an amount of the one or more probiotic bacteria and fermentate effective to provide a health benefit to a subject. In some aspects, the effective amount is a therapeutically effective amount.

In some aspects, a composition can be formulated such that the one or more of the bacteria present in the composition can replicate once they are delivered to the target habitat (e.g., the gut). In one non-limiting example, the composition is formulated in a pill, powder, capsule, tablet, enteric-coated dosage form or package, such that the composition has a shelf life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In some aspects, other components are added to the composition to aid in the shelf life of the composition. In some aspects, one or more bacteria may be formulated in a manner allowing survival in a non-natural environment. For example, bacteria that is native to the gut may not survive in an oxygen-rich environment. To overcome this limitation, the bacteria may be formulated in a pill or package that can reduce or eliminate the exposure to oxygen. Other strategies to enhance the shelf-life of bacteria may include other microbes (e.g., if the bacterial consortia comprise a composition whereby one or more strains are helpful for the survival of one or more strains).

In some aspects, the composition can be formulated as a powder, tablet, capsule, enteric-coated dosage form (e.g., for delivery to ileum/colon), or pill that can be administered to a subject by any suitable route. The lyophilized formulation can be mixed with a saline or other solution prior to administration.

In some aspects, the composition is formulated for oral administration. In some aspects, the composition is formulated as a powder, tablet, capsule, enteric-coated dosage form or pill for oral administration. In some aspects, the composition is formulated for delivery of the bacteria to the ileum region of a subject. In some aspects, the composition is formulated for delivery of the bacteria to the colon region (e.g., upper colon) of a subject. In some aspects, the composition is formulated for delivery of the bacteria to the ileum and colon regions of a subject.

An enteric coating can protect the contents of the oral formulation, for example, tablet or capsule, from the acidity of the stomach and provide delivery to the ileum and/or upper colon regions. Non-limiting examples of enteric coatings include pH sensitive polymers (e.g., Eudragit® FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. In some aspects, the enteric coating is formed by a pH sensitive polymer. In some aspects, the enteric coating is formed by Eudragit® FS30D.

In some aspects, the enteric coating can be designed to dissolve at any suitable pH. In some aspects, the enteric coating is designed to dissolve at a pH greater than about pH 5.0, or at a pH greater than about pH 6.0, or at a pH greater than about pH 7.0. In some aspects, the enteric coating is designed to dissolve at a pH greater than about pH 5.0 to about pH 7.0. In some aspects, the enteric coating is designed to dissolve at a pH greater than about pH 5.0, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5.

Formulations provided herein can include the addition of one or more agents to the composition in order to enhance stability and/or survival of the microbial formulation. Non-limiting example of stabilizing agents can include genetic elements, glycerin, ascorbic acid, skim milk, lactose, tween, alginate, xanthan gum, carrageenan gum, mannitol, palm oil, poly-L-lysine (POPL), and combinations thereof.

In some aspects, the composition can be formulated into unit dosage form, i.e., in the form of discrete portions containing a unit dose, or a multiple dose, or a sub-unit of a unit dose. For example, a typical or usual suitable or effective dose in humans of the one or more bacteria is from about 1×E3 (1×E3=1×10\hat{ }3=1×(10 to the power 3)) to about 1×E13 colony forming units (CFU). In some instances, a suitable or effective dose is from about 1×E6 to about 1×E11 CFU. In particular instances, a suitable or effective dose is from about 1×E7 to about 1×E10 CFU. In some additional aspects, a suitable or effective dose of the bacteria can be about 1×E2 CFU, 1×E3 CFU, 1×E4 CFU, 1×E5 CFU, 1×E6 CFU, 1×E7 CFU, 1×E8 CFU, 1×E9 CFU, 1×E10 CFU, 1×E11 CFU, 1×E12 CFU, 1×E13 CFU, 1×E14 CFU, or 1×E15 CFU.

The composition can be administered once daily. Alternatively, the composition can be taken twice daily, alternatively three times daily, alternatively four times daily. The composition can be taken with meals or on an empty stomach. The composition can be taken in the morning, mid-day, afternoon, evening, or at night. The composition can be taken at the same time every day or the time the composition is taken can vary. A user can administer one dosage form per dose of the composition, in another example two dosage forms, in another example three dosage forms, in another example four dosage forms, and in another example more than four dosage forms. In some aspects, the dose is about 0.1 milligrams (mg), about 0.2 mg, about 0.3 mg, about 0.4 mg, about 0.5 mg, about 0.6 mg, about 0.7 mg, about 0.8 mg, about 0.9 mg, about 1.0 mg, about 2.0 mg, about 3.0 mg, about 4.0 mg, about 5.0 mg, about 6.0 mg, about 7.0 mg, about 8.0 mg, about 9.0 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 300 mg, about 400 mg, about 500 mg, about 600 mg, about 700 mg, about 800 mg, about 900 mg, or about 1 gram. In some aspects, a dose ranges from about 1 mg to about 500 mg.

In some aspects, the composition can comprise from about 5 to about 10 mg of IPA per dose of the composition. Through pharmacokinetic studies, it was found that a dose of from about 5 to about 10 mg of IPA can be sufficient to increase IPA levels in serum beyond endogenous baseline values.

In some aspects, the composition can comprise a prebiotic and a dose of the composition can be from about 50 mg to about 5 g, alternatively from about 100 mg to about 4 g, alternatively from about 250 mg to about 2 g.

In some aspects, the composition can comprise one or more bacteria in an amount of from about 1×E3 to about 1×E13 colony forming units (CFU)/gram (g), with respect to the weight of the composition. In some aspects, one or more bacteria are present in an amount from about 1×E5 to about 1×E11 CFU/g. In some aspects, one or more bacteria are present in an amount from about 1×E6 to about 1×E10 CFU/g. In some aspects, one or more bacteria are present in the composition in an amount from about 1×E8 to about 1×E10 CFU/g. In some aspects, a composition comprises one or more bacteria present in an amount of about 1×E1 CFU/g, about 1×E2 CFU/g, about 1×E3 CFU/g, about 1×E4 CFU/g, about 1×E5 CFU/g, about 1×E6 CFU/g, about 1×E7 CFU/g, about 1×E8 CFU/g, about 1×E9 CFU/g, about 1×E10 CFU/g, about 1×E11 CFU/g, about 1×E12 CFU/g, about 1×E13 CFU/g, about 1×E14 CFU/g, or about 1×E15 CFU/g.

Suitable containers for use with the composition described herein include, for example, cans, jars, bottles, bottles with shaker lids, mills, vials, syringes, tubes, pouches, sachets, bags, blister cards, or folders. The containers can be formed from a variety of materials including without limitation glass, plastic, polymers, metals, alloys, metal or alloy foil, rubber, cardboard, or paper. The containers can also comprise a sealant, which can be formed from any material suitable in the art such as a resin or polymer. The container can comprise a moisture barrier and/or oxygen barrier to further enhance the viability of the probiotics during storage. Moisture barriers and oxygen barriers are known in the pharmaceutical and food industries. Suitable barriers for use in the present invention are described in U.S. Pat. No. 6,716,499 to Vadhar, U.S. Pat. No. 6,524,720 to Shah, U.S. Pat. No. 5,792,530 to Bonner et al., and U.S. Pat. No. 4,977,004 to Bettie et al. In addition to, or in lieu of such barriers, the containers may comprise an oxygen scavenger and/or a desiccant/moisture absorbing compound. Suitable oxygen scavengers and desiccants are known in the art, for example, U.S. Pat. No. 6,746,622 to Yan et al., U.S. Pat. No. 6,387,461 to Ebner et al., and U.S. Pat. No. 6,228,284 to Ebner et al., and U.S. Pat. No. 6,130,263 to Hekal.

Also described herein are methods of providing one or more health benefits comprising orally administering the present composition to a user. In some aspects, the one or more health benefits may be selected from the group consisting of promoting brain health; promoting healthy aging of the brain; promoting emotional well-being via brain health; delivering antioxidant nutrients to the brain; managing oxidative stress in the brain; reducing and/or maintaining oxidative stress or total antioxidant capacity in the brain; protecting neurons via delivering antioxidants; and any combination of the foregoing. In some aspects, the one or more health benefits may be selected from the group consisting of promoting brain health; promoting healthy aging of the brain; delivering antioxidant nutrients to the brain; managing oxidative stress in the brain; and any combination of the foregoing.

Also described herein are methods of increasing IPA in the gastrointestinal tract and/or serum of a subject in need thereof comprising administering to the subject an effective amount of the composition described herein.

Also described herein are methods for optimizing the gut-brain axis for a healthy nervous system via reducing neuroinflammation and neurodegeneration of a subject in need thereof comprising administering to the subject an effective amount of the composition described herein.

Also described herein are methods for treating, ameliorating, or preventing a disorder in a subject suffering therefrom or at risk of suffering therefrom comprising administering to the subject an effective amount of the composition described herein. In some aspects, the disorder can be an intestinal disorder, a metabolic disorder, an inflammatory disorder, or an immune disorder. In some aspects, the disorder can be a metabolic syndrome, insulin resistance, insulin sensitivity, pre-diabetes, diabetes, anxiety, depression, autism, hypertension, irritable bowel syndrome, metabolism irregularity, stress-related conditions, neurological disorders, such as Parkinson's disease, Inflammatory Bowel Disease (IBD), Crohn's Disease, heart disease, or a nervous system disorder such as multiple sclerosis.

### EXAMPLES AND DATA

The following data and examples are provided to help illustrate the invention described herein. The exemplified compositions are given solely for the purpose of illustration and are not to be construed as limitations of the present invention, as many variations thereof are possible without departing from the spirit and scope of the invention. All parts, percentages, and ratios herein are by weight unless otherwise specified.

Production of IPA and Derivatives Thereof

A fermentation medium was first prepared according to the formula in Table 2.

TABLE 2
Fermentation Medium 1

| Ingredient | Amt. Per 1 L | |
|---|---|---|
| Resazurin (0.01% w/v stock) | 1.00 | ml |
| K2HPO4 | 2.00 | g |
| KH2PO4 | 2.00 | g |
| MgCl2 6H2O | 0.20 | g |
| (NH4)2SO4 | 5.00 | g |
| NaHCO3 (10% w/v stock) | 25.00 | mL |
| Cysteine•HCl (5% w/v stock) | 10.00 | mL |
| L-glycine | 0.0751 | g |
| L-valine | 0.1172 | g |
| L-leucine | 0.1312 | g |
| L-isoleucine | 0.1312 | g |
| L-methionine | 0.1492 | g |
| L-histidine | 0.1552 | g |
| L-arginine | 0.1742 | g |
| L-phenylalanine | 0.1652 | g |
| L-tyrosine | 0.1812 | g |
| L-tryptophan | 0.2042 | g |
| Trace Element Solution¹ | 10.00 | mL |
| Vitamin Solution² | 10.00 | mL |

| | |
|---|---|
| Demineralized Water | Q.S. |

¹Trace Mineral Supplement ATCC ® MD-TMS ™ (commercially available from ATCC, Manassas, VA).
²Vitamin Supplement ATCC ® MD-VS ™ (commercially available from ATCC, Manassas, VA).

All ingredients, except the amino acids, Trace Element Solution and Vitamin Solution were combined and heated with agitation to 121° C. for 30-40 minutes. The mixture was allowed to cool for about 10-20 minutes before the amino acids, Trace Element Solution and Vitamin Solution were added. Prior to adding the amino acids, an amino acid solution was prepared by dissolving all of the amino acids listed in Table 2 in a 100 mL aliquot of cooled media and filter sterilizing.

Clostridium sporogenes ATCC 15579 (C. sporogenes) was grown anaerobically at 36° C. for 24 hours in 10 mL of Peptone Yeast Glucose (“PYG”) media (commercially available from Sigma-Aldrich, St. Louis, Mo.). A 10 mL aliquot of the 24 hr culture (approximately 1×E8 CFU/mL) was centrifuged at 10,0000×g for 5 min. The supernatant was removed and the C. sporogenes pellet was resuspended in 10 mL of saline to wash the bacteria. The sample was then centrifuged at 10,000×g for 5 min. The supernatant was removed and the C. sporogenes pellet was resuspended in 10 mL of saline to create an inoculum preparation.

100 μl of the inoculum preparation was transferred anaerobically into 10 mL of fermentation medium in replicates. After preparation, the tubes were transferred into a 36° C. box in an anaerobic chamber for 24-28 hrs. After incubation, the tubes were removed from the chamber and were centrifuged at 8,000×g for 10 minutes. The supernatant was removed and was filtered through a 0.2 μm syringe filter into a sterile glass tube.

Then, a 3 mL aliquot of the supernatant was transferred into a lyophilization vial. 5% Food Grade Hi-Cap® 100 starch (commercially available from Ingredion, Westchester, Ill.) was added to the vial. The sample was then lyophilized. First, in the thermal treatment step, the vials were held at 20° C. for 30 minutes, followed by 0° C. for 80 minutes, and then at −25° C. for 60 minutes. The freezing was performed at −25° C. (condenser at −50° C.) and vacuum at 200 mTorr. Next, in the drying step, the vials were held at −25° C. for a total of about 1800 to about 1830 minutes at 200 mTorr and then the temperature was ramped up to 4° C. and vials were held at 4° C. for 60 minutes at 200 mTorr. After lyophilization, the fermentate powder was subjected to analytical measurement of IPA according to the IPA Measurement Method described hereafter. 150 uM pure IPA in fermentation medium was used as a control. The results are set forth below in Table 3.

TABLE 3

| | Amount | |
|---|---|---|
| Constituent | (mg/g fermentate) | St. Dev |
| Indole-3-Propionic acid | 0.308 | 0.011 |
| Indole-3-Acrylic Acid | 0.002 | 0.000 |
| Tryptophan | 0.492 | 0.020 |
| Indole-3-Lactic Acid | 0.058 | 0.002 |

It was found that IPA and derivatives thereof can be produced via bacterial fermentation in a defined fermentation medium and lyophilization of the supernatant. IPA, indole-3 acrylic acid, indole-3-lactic acid, and tryptophan could be detected in the fermentate powder. It was found that starch did not inhibit the recovery of IPA.

### Fermentation Media

After demonstrating in a proof of concept that IPA and other indole derivatives could be produced via bacterial fermentation (Table 3), different fermentation media were tested to assess whether the level of IPA and other derivatives production could be increased. Fermentation Medium A contained the same formula as Medium 1 with the addition of glucose, Fermentation Medium B was the same formula as Medium A but contained tryptophan as the only amino acid, and Fermentation Medium C contained the same formula as Media 1 but with 10× the amino acids. Fermentation Media A, B, and C were prepared according to the formulas in Table 4. These media were compared to Fermentation Medium 1 described above in Table 2.

TABLE 4
Fermentation Media

| Ingredient | Medium A | | Medium B | | Medium C | |
|---|---|---|---|---|---|---|
| Resazurin (0.01% w/v stock) | 1.00 | mL | 1.00 | mL | 1.00 | mL |
| K2HPO4 | 2.00 | g | 2.00 | g | 2.00 | g |
| KH2PO4 | 2.00 | g | 2.00 | g | 2.00 | g |
| MgCl2 6H2O | 0.20 | g | 0.20 | g | 0.20 | g |
| (NH4)2SO4 | 5.00 | g | 5.00 | g | 5.00 | g |
| NaHCO3 (10% w/v stock) | 25.00 | mL | 25.00 | mL | 25.00 | mL |
| Cysteine•HCl (5% w/v stock) | 10.00 | mL | 10.00 | mL | 10.00 | mL |
| L-glycine | 0.0751 | g | 0 | | 0.7510 | g |
| L-valine | 0.1172 | g | 0 | | 1.1720 | g |
| L-leucine | 0.1312 | g | 0 | | 1.3120 | g |
| L-isoleucine | 0.1312 | g | 0 | | 1.3120 | g |
| L-methionine | 0.1492 | g | 0 | | 1.4920 | g |
| L-histidine | 0.1552 | g | 0 | | 1.5520 | g |
| L-arginine | 0.1742 | g | 0 | | 1.7420 | g |
| L-phenylalanine | 0.1652 | g | 0 | | 1.6520 | g |
| L-tyrosine | 0.1812 | g | 0 | | 1.8120 | g |
| L-tryptophan | 0.2042 | g | 0.2042 | g | 2.0423 | g |
| Trace Element Solution¹ | 10.00 | mL | 10.00 | mL | 10.00 | mL |
| Vitamin Solution² | 10.00 | mL | 10.00 | mL | 10.00 | mL |

| | | | | | |
|---|---|---|---|---|---|
| Glucose Stock (500 mM) | 40 | mL | 40.00 | mL | 0 |

| | | | |
|---|---|---|---|
| Demineralized Water | Q.S. | Q.S. | Q.S. |

¹Trace Mineral Supplement ATCC ® MD-TMS ™ (commercially available from ATCC, Manassas, VA).
²Vitamin Supplement ATCC ® MD-VS ™ (commercially available from ATCC, Manassas, VA).

All ingredients, except the amino acids, glucose stock solution, Trace Element Solution and Vitamin Solution were combined and heated with agitation to 121° C. for 30-40 minutes. The mixture was allowed to cool for about 10-20 minutes before the amino acids, glucose stock solution, Trace Element Solution and Vitamin Solution were added. Prior to adding the amino acids, an amino acid solution was prepared by dissolving all of the amino acids listed in Table 4 in a 100 mL aliquot of cooled media and filter sterilizing.

Clostridium sporogenes ATCC 15579 (C. sporogenes) was grown anaerobically at 36° C. for 24 hours in 10 mL of Peptone Yeast Glucose (“PYG”) media (commercially available from Sigma-Aldrich, St. Louis, Mo.). A 10 mL aliquot of the 24 hr culture (approximately 1×E8 CFU/mL) was centrifuged at 10,0000×g for 5 min. The supernatant was removed and the C. sporogenes pellet was resuspended in 10 mL of saline to wash the bacteria. The sample was then centrifuged at 10,000×g for 5 min. The supernatant was removed and the C. sporogenes pellet was resuspended in 10 mL of saline to create an inoculum preparation.

100 μl of the inoculum preparation was transferred anaerobically into 10 mL of fermentation medium in replicates. After preparation, the tubes were transferred into a 36° C. box in an anaerobic chamber for 24-28 hrs. After incubation, the tubes were removed from the chamber and were centrifuged at 8,000×g for 10 minutes. The supernatant was removed and was filtered through a 0.2 μm syringe filter into a sterile glass tube. The supernatant was then subjected to analytical measurement of IPA according to the IPA Measurement Method described hereafter. The results are set forth below in Table 5.

TABLE 5

| | Indole-3- | | | |
|---|---|---|---|---|
| | propionic | Indole-3- | Indole-3- | Indole-3- |
| | acid | acrylic acid | acetic acid | lactic acid |
| Media | μg/mL | | | |
| Fermentation | 42.4 ± 3.0 | 0.91 ± 0.06 | 0.11 ± 0.007 | 6.00 ± 0.39 |
| Medium 1 | | | | |
| Fermentation | 127.3 ± 6.4 | 3.00 ± 0.07 | 0.11 ± 0.007 | 12.4 ± 1.27 |
| Medium A | | | | |
| Fermentation | 22.85 ± 2.96 | Not detected | Not detected | 1.29 ± 0.35 |
| Medium B | | | | |
| Fermentation | 0.62 ± 0.06 | 0.12 ± 0.007 | Not detected | 1.23 ± 0.12 |
| Medium C | | | | |

It was surprisingly found that the addition of glucose to the growth medium (Fermentation Medium A) can significantly improve the IPA yield as compared to growth medium without glucose (Fermentation Medium 1). Growth medium containing glucose and tryptophan as the only amino acid (Fermentation Medium B) reduced the IPA and derivative yield. It was also found that increasing the concentration of amino acids (Fermentation Medium C) significantly lowered the IPA as compared to Fermentation Medium 1.

### EXAMPLES

| | Example 1 | Example 2 | Example 3 | Example 4 | Example 5 |
|---|---|---|---|---|---|
| Ingredient | Wt % | Wt % | Wt % | Wt % | Wt % |
| Fermentate | 75.0 | 60.0 | 65.0 | 70.0 | 75.0 |
| Micro- | 10.0 | 30.0 | 33.5 | 29.0 | 24.0 |
| crystalline | | | | | |
| Cellulose | | | | | |
| Maltodextrin | 14.5 | 9.0 | 0 | 0 | 0 |
| Magnesium | 0.5 | 1.0 | 1.5 | 1.0 | 1.0 |
| Stearate | | | | | |

| | Example 6 | Example 7 | Example 8 | Example 9 |
|---|---|---|---|---|
| Ingredient | Wt % | Wt % | Wt % | Wt % |
| Fermentate | 60.0 | 65.0 | 85.0 | 80.0 |
| Micro- | 30.0 | 9.0 | 14.0 | 10.0 |
| crystalline | | | | |
| Cellulose | | | | |
| Maltodextrin | 9.0 | 25.0 | 0 | 9.0 |
| Magnesium | 1.0 | 1.0 | 1.0 | 1.0 |
| Stearate | | | | |

Examples 1-9 can be made according to the following method.

Fermentation Media Preparation

Fermentation Medium 1 can be prepared by combining all the ingredients listed in Table 2, except the amino acids, Trace Element Solution, and Vitamin Solution. The medium can be sterilized by heating with agitation to 121° C. for 30-40 minutes. The medium can then be cooled for about 10-20 minutes before the amino acids, Trace Element Solution, and Vitamin Solution are added. Prior to adding the amino acids, an amino acid solution can be prepared by dissolving the amino acids in a 100 mL aliquot of cooled media and filter sterilizing. The media can then be handled and stored under sterile conditions until use.

Alternatively, the fermentate in Examples 1-9 can be prepared using Fermentation Medium A. Fermentation Medium A can be prepared by combining all the ingredients listed in Table 4, except the amino acids, glucose stock solution, Trace Element Solution, and Vitamin Solution. The medium can be sterilized by heating with agitation to 121° C. for 30-40 minutes. The medium can then be cooled for about 10-20 minutes before the amino acids, glucose stock solution, Trace Element Solution, and Vitamin Solution are added. Prior to adding the amino acids, an amino acid solution can be prepared by dissolving the amino acids in a 100 mL aliquot of cooled media and filter sterilizing. The media can then be handled and stored under sterile conditions until use.

Bacteria Preparation

Clostridium sporogenes ATCC 15579 (C. sporogenes) can be grown anaerobically at 36° C. in Peptone Yeast Glucose (“PYG”) media (commercially available from Sigma-Aldrich, St. Louis, Mo.). An aliquot of the 24 hour culture (approximately 1×E8 CFU/mL) can be centrifuged to produce a pellet. The supernatant can be removed and the C. sporogenes pellet can be resuspended in saline to wash the bacteria. The sample can then be centrifuged again. The supernatant can be removed and the C. sporogenes pellet resuspended in saline to create an inoculum preparation.

Fermentate Preparation

The C. sporogenes inoculum preparation can be transferred anaerobically into the fermentation medium to form a bacteria solution. The fermentation can be carried out in a suitably sized fermenter at 36° C. until maximum growth is achieved.

To make a fermentate in which the bacteria are removed, the fermented bacteria solution can be centrifuged to remove the bacteria and the supernatant can be collected. Optionally, the supernatant can be passed through a combination of reverse osmosis, tray drying, microfiltration, and nanofiltration to reduce the water content prior to drying. The resulting concentrated liquid can be mixed with mannitol and starch as dehydrating agents. The supernatant can then be spray dried to produce powdered fermentate containing IPA. Alternatively, the supernatant can be sprayed into liquid nitrogen to produce frozen beads. The frozen beads can be dried by lyophilization followed by milling to produce powdered fermentate containing IPA.

To make a fermentate that contains bacteria, the fermented bacteria solution can be inactivated by heating or by treatment with a proteolytic enzyme. The resulting solution can be mixed with mannitol and starch as dehydrating agents. The solution can then be spray dried to produce powdered fermentate containing IPA. Alternatively, the solution can be sprayed into liquid nitrogen to produce frozen beads. The frozen beads can be dried by lyophilization followed by milling to produce powdered fermentate containing IPA.

The powdered fermentate containing IPA can be weighed and loaded into a powder blender, such as a suitably sized “V” blender. Microcrystalline cellulose (USP) and maltodextrin (USP) (if present in the formula) can be separately sieved, weighed, and loaded into the powder blender. Blending can be carried out until a homogeneous blend of fermentate and excipients is obtained, typically mixing can be carried out for 100-500 revolutions. Magnesium stearate (USP) can be sieved and loaded into the powder blender. The magnesium stearate can be incorporated into the fermentate powder by blending for typically less than 100 rotations.

The final blend can be loaded into the powder feed hopper of a rotary encapsulator equipped with a capsule polisher. Gelatin or hydroxypropylmethyl cellulose capsules can be loaded into the capsule hopper. Capsules can be filled with the final blend and polished. Alternatively, the final blend can be loaded into a sachet filler equipped with a sachet sealer and the sachet material can be loaded. Sachets can be filled and sealed.

### IPA Measurement Method

Biological samples were subjected to protein precipitate by adding 300 μL of MeOH to 100 μL of sample. Samples were vortexed and centrifuged for 10 minutes at 3000 rpm using a benchtop centrifuge such as a Beckman Coulter Allegra® X 15R (Rotor SX4750A), or equivalent, to pellet the protein and other precipitates. 150 μL of supernatant was transferred to a 96-well deep well plate along with 30 μL of 10 ng/mL Indole-3-Propionic Acid-2,2-d2 (IPA-d2) and 150 μL of water. For samples in other matrices including, but not limited to, bacterial cell culture filtrates and fermentates, samples were subjected to 1000-fold dilution with 10% MeOH in water. 30 μL of 10 ng/mL IPA-d2 were added to 300 μL of the diluted sample. The IPA and IPA-d2 in the isolated/diluted samples were subjected to gradient High-Performance Liquid Chromatography (HPLC) analysis on a Waters Atlantis T3 column, from Waters Corp., Milford, Mass., or equivalent, (2.1×50 mm, 3 μm particles), 0.1% formic acid in Water as mobile phase A and 0.1% formic acid in acetonitrile as mobile phase B. Detection and quantitation were achieved by tandem mass spectrometry operating under multiple reaction monitoring (MRM) MS/MS conditions (m/z 190.1130.0 for IPA, m/z 192.1→130.0 for IPA-d2). IPA calibration standards (STD), prepared in 10% MeOH in water, were used to construct a regression curve by plotting the response (peak area IPA/peak area IPA-d2) versus concentration for each standard. The concentrations of IPA in samples were determined by interpolation from the quadratic (1/x²) regression curve.

### Combinations

A. A method of producing indole-3-propionic acid and other indole derivatives comprising: adding bacteria having a nucleic acid sequence with at least 80% homology to the nucleic acid sequence of SEQ ID NO: 1 to a liquid fermentation medium to form a bacteria solution; fermenting the bacteria solution at 36° C. under anaerobic conditions; adding a dehydrating agent; and dehydrating to obtain a fermentate powder comprising indole-3-propionic acid and other indole derivatives.
B. The method according to paragraph A, further comprising centrifuging the fermented bacteria solution to form a supernatant solution and a bacteria pellet; removing the supernatant solution; and adding the dehydrating agent to the supernatant solution.
C. The method according to paragraph A or B, wherein the fermentate powder comprises from 0.1 to 20 mg/g indole-3-propionic acid.
D. The method according to any of paragraphs A-C, wherein the fermentate powder comprises from 0.1 to 20 mg/g other indole derivatives.
E. The method according to any of paragraphs A-D, wherein the fermentation medium comprises water, an amino acid composition, a salt, a mineral, and optionally a carbohydrate.
F. The method according to any of paragraphs A-E, further comprising homogenizing prior to the dehydrating step.
G. The method according to any of paragraphs A-F, wherein the dehydrating step is spray-drying.
H. The method according to any of paragraphs A-G, wherein the dehydrating step is freeze-drying.
I. A composition comprising: a fermentate comprising bacterially derived indole-3-propionic acid; and an excipient, carrier, and/or diluent.
J. The composition of paragraph I, wherein the composition comprises from 0.01 to 10% indole-3-propionic acid, by weight of the composition.
K. The composition of paragraphs I or J, wherein the composition further comprises an indole derivative selected from the group consisting of indole-3-acetic acid, indole-3-acrylic acid, indole-3-lactic acid, and combinations thereof.
L. The composition of any of paragraphs I-K, wherein the composition further comprises one or more bacteria selected from the group consisting of Bifidobacterium bifidum, Bifidobacterium breve, Bifidobacterium infantis, Bifidobacterium longum, Streptococcus cremoris, Streptococcus diacetylactis, Streptococcus lactis, Streptococcus thermophilus, Lactobacillus acidophilus, Lactobacillus bifidus, Lactobacillus bulgaricus, Lactobacillus casei, Lactobacillus delbruekii, Lactobacillus crispatis, Lactobacillus fermentii, Lactobacillus gasseri, Lactobacillus helveticus, Lactobacillus johnsonii, Lactobacillus lactis, Lactobacillus plantarum, Lactobacillus rhamnosus, Lactobacillus paracasei, Lactobacillus reuteri, Lactobacillus salivarius, Lactobacillus thermophilus, Lactococcus lactis, Clostridium sporogenes, Peptostreptococcus anaerobius, Clostridium cadaveris, Clostridium boltae, and combinations thereof.
M. The composition of paragraph L comprising from 1×E3 to 1×E11 colony-forming units (CFU) of the one or more bacteria.
N. The composition of any of paragraphs I-M, wherein the fermentate further comprises tryptophan.
O. A method of promoting brain health comprising administering to an individual in need thereof the composition of paragraph I.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Values disclosed herein as ends of ranges are not to be understood as being strictly limited to the exact numerical values recited. Instead, unless otherwise specified, each numerical range is intended to mean both the recited values and any real numbers including integers within the range. For example, a range disclosed as “1 to 10” is intended to mean “1, 2, 3, 4, 5, 6, 7, 8, 9, and 10” and a range disclosed as “1 to 2” is intended to mean “1.1, 1.2, 1.3, 1.4, 1.5, 1.6, 1.7, 1.8, 1.9, and 2.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising:
a. a fermentate comprising bacterially derived indole-3-propionic acid and other indole derivatives; and
b. an excipient, carrier, and/or diluent.

2. The composition of claim 1, wherein the composition comprises from about 0.01 to about 10% indole-3-propionic acid, by weight of the composition.

3. The composition of claim 1, wherein the composition comprises from about 0.1 to about 20 mg/g indole-3-propionic acid.

4. The composition of claim 1, wherein the composition comprises from about 0.01 to about 10% other indole derivatives, by weight of the composition.

5. The composition of claim 4, wherein the other indole derivatives are selected from the group consisting of indole-3-acetic acid, indole-3-acrylic acid, indole-3-lactic acid, and combinations thereof.

6. The composition of claim 1, further comprising one or more bacteria selected from the group consisting of Bifidobacterium bifidum, Bifidobacterium breve, Bifidobacterium infantis, Bifidobacterium longum, Streptococcus cremoris, Streptococcus diacetylactis, Streptococcus lactis, Streptococcus thermophilus, Lactobacillus acidophilus, Lactobacillus bifidus, Lactobacillus bulgaricus, Lactobacillus casei, Lactobacillus delbruekii, Lactobacillus crispatis, Lactobacillus fermentii, Lactobacillus gasseri, Lactobacillus helveticus, Lactobacillus johnsonii, Lactobacillus lactis, Lactobacillus plantarum, Lactobacillus rhamnosus, Lactobacillus paracasei, Lactobacillus reuteri, Lactobacillus salivarius, Lactobacillus thermophilus, Lactococcus lactis, Clostridium sporogenes, Peptostreptococcus anaerobius, Clostridium cadaveris, Clostridium boltae, and combinations thereof.

7. The composition of claim 6 comprising from about 1×E3 to about 1×E11 colony-forming units (CFU) of the one or more bacteria.

8. The composition of claim 1, wherein the fermentate further comprises tryptophan.

9. The composition of claim 1, wherein the composition further comprises an active ingredient.

10. The composition of claim 1, wherein the composition further comprises an herbal ingredient.

11. The composition of claim 8, wherein the fermentate comprises from about 0.1 mg/g to about 0.5 mg/g tryptophan.

12. A method of promoting brain health comprising administering to an individual in need thereof the composition of claim 1.

13. A method of delivering antioxidant nutrients to the brain comprising administering to an individual in need thereof the composition of claim 1.

14. A method of producing indole-3-propionic acid and other indole derivatives comprising:
a. adding bacteria having a nucleic acid sequence with at least 80% homology to the nucleic acid sequence of SEQ ID NO: 1 to a liquid fermentation medium to form a bacteria solution;
b. fermenting the bacteria solution at about 36° C. under anaerobic conditions;
c. adding a dehydrating agent; and
d. dehydrating to obtain a fermentate powder comprising indole-3-propionic acid and other indole derivatives.

15. The method of claim 14, wherein the fermentation medium comprises water, an amino acid composition, a salt, a mineral, and a carbohydrate.

16. The method of claim 15, further comprising homogenizing prior to the dehydrating step.

17. The method of claim 15, wherein the dehydrating step is spray-drying.

18. The method of claim 15, wherein the dehydrating agent is starch.

19. The method of claim 15, further comprising centrifuging the fermented bacteria solution to form a supernatant solution and a bacteria pellet; removing the supernatant solution; and adding the dehydrating agent to the supernatant solution.

20. The method of claim 15, wherein the fermentate powder comprises from about 0.1 to about 20 mg/g indole-3-propionic acid.
